(19) United States
(12) Patent Application Publication
(10) Pub. No.: US 2023/0398129 A1
(43) Pub. Date: Dec. 14, 2023

(54) CRYSTAL-FREE HIGH-CONCENTRATION TESTOSTERONE CYPIONATE FORMULATIONS

(71) Applicant: Pfizer Inc., New York, NY (US)

(72) Inventor: Christopher Lee Beltz, Portage, MI (US)

(73) Assignee: Pfizer Inc, New York, NY (US)

(21) Appl. No.: 18/250,570

(22) PCT Filed: Oct. 26, 2021
(86) PCT No.: PCT/IB2021/059886

§ 371 Date: Apr. 26, 2023

Related U.S. Application Data
(60) Provisional application No. 63/107,185, filed on Oct. 29, 2020.

(51) Int. Cl. A61K 31/569; A61K 47/14; A61K 47/10; A61K 47/44; A61K 9/08; A61K 9/00

(52) U.S. Cl. CPC A61K 31/569; A61K 47/14; A61K 47/10; A61K 47/44; A61K 9/08; A61K 9/0019

(57) ABSTRACT

A pharmaceutical formulation for parental use comprising testosterone cypionate with a concentration of 200 mg/mL, benzyl benzoate with a concentration of 25%-35% v/v, cottonseed oil, and a preservative. A pharmaceutical product comprising a vial containing said formulation. A kit which comprises said pharmaceutical product and a package insert that is free of instructions to warm and shake the vial.

### BACKGROUND OF THE INVENTION

Commercially available parental formulations of testosterone cypionate, such as DEPO-TESTOSTERONE™, that use high concentrations of testosterone have the potential to form crystals in the product during storage. In particular, the 200 mg/mL solution is highly concentrated (i.e., supersaturated) with respect to the active pharmaceutical ingredient (API). The high concentration of API makes the product susceptible to crystallization when exposed to a temperature lower than recommended on the label [20° C. to 25° C. (68° F. to 77° F.)]. This product attribute is well known and is specifically addressed in both the U.S. package insert and on the product vial label via inclusion of the following statement: “Warming and shaking the vial should re-dissolve any crystals that may have formed during storage at temperatures lower than recommended.” DEPO-TESTOSTERONE™ [package insert] New York, NY: Pfizer, Inc.; revised July 2018.

A least since 2011, it has been known that crystals may form in testosterone cypionate products. The presence of crystals may impact visible appearance and quality control as it may impair the ability to inspect vials to detect foreign material. While the cause of crystallization is known, a solution to this issue has yet to be presented for over 10 years as there exists no vial product on market today that is not susceptible to crystallization, leaving a long felt need to address this issue.

In order to address the aforementioned issue, novel formulations described herein eliminate the known crystallization issues, yet maintain true solution properties. Moreover, it should be noted that the novel formulations of the invention have not significantly altered the physical and chemical properties of the drug-in-oil solution formulation. These crystal-free formulations allow for improved inspectability, a reduction in the number of vials returned as defective, and a reduction in preparation time to administer the product, offering a key advantage of time saving over competitor products. Another competitor advantage is a potential opportunity to user convenience.

### SUMMARY OF THE INVENTION

The present invention provides a new pharmaceutical formulation for parental use comprising testosterone cypionate with a concentration of 200 mg/mL, benzyl benzoate with a concentration of 25%-35% v/v, cottonseed oil, and a preservative. The present invention eliminates the known crystallization problems without significantly altering the physical and chemical properties of the formulation. These improved formulations will also reduce preparation time to administer the product.

### BRIEF DESCRIPTION OF THE DRAWINGS

FIG. 1 provides a comparison of solubility as a function of temperature for the formulations claimed herein compared to the existing commercial product.

### DETAILED DESCRIPTION OF THE INVENTION

According to a first aspect of the invention, there is provided a pharmaceutical formulation for parental use comprising testosterone cypionate with a concentration of 200 mg/mL, benzyl benzoate with a concentration of 25%-35% v/v, cottonseed oil, and a preservative. Described below are a number of embodiments (E) of this first aspect of the invention, where for convenience E1 is identical thereto.

E1. The formulation, according to the first aspect of the invention, as set out just above.

E2. The formulation according to embodiment E1, wherein the benzyl benzoate concentration is 30% v/v, the cottonseed oil has a concentration of 460-480 mg/mL, and the preservative is benzyl alcohol with a concentration of 9 to 10 mg/mL.

E3. The formulation according to embodiment E2 wherein the cottonseed oil has a concentration of 470 mg/mL, and the benzyl alcohol has a concentration of 9.45 mg/mL.

E4. The formulation according to embodiment of E3 wherein the formulation has a volume of 1 mL.

E5. The formulation according to embodiment E4, consisting essentially of 200 mg of testosterone cypionate, 0.3 mL benzyl benzoate, 470 mg of cottonseed oil, and 9.45 mg benzyl alcohol.

E6. The formulation of any of the embodiments E1 to E5, wherein the formulation is free of visible crystals when stored at 20° C.

E7. A pharmaceutical product comprising a vial containing a formulation according to any one embodiments E1 to E6.

E8. A kit which comprises the pharmaceutical product according to embodiment E7 and a package insert that is free of instructions to warm and shake the vial.

E9. A method for treating a deficiency or absence of endogenous testosterone in a subject, comprising administering to the subject in need of such treatment a therapeutically effective amount of the formulation according to any one of the embodiments E1 to E6.

E10. A method for treating primary hypogonadism testicular failure, bilateral torsion, orchitis, vanishing testis syndrome, orchidectomy, hypogonadotropic hypogonadism gonadotropin, LHRH deficiency, or pituitary-hypothalamic injury in a subject, comprising administering to the subject in need of such treatment a therapeutically effective amount of the formulation according to any one of the embodiments E1 to E6.

E11. A formulation as defined in any of the embodiments E1 to E6, for use as a medicament.

E12. A formulation as defined in any of the embodiments E1 to E6, for use in a method of treating any of the diseases recited in embodiment E10.

E13. The use of a formulation as defined in any of the embodiments E1 to E6 in the manufacture of a medicament for use in treating any of the diseases recited in embodiment E10.

### Definitions

“200 mg” or “200 mg/mL” of testosterone cypionate, as used herein, includes an overage in an amount of up to 10 mg (or 5% by weight), for example up to 210 mg, for minor overages of testosterone cypionate during filing. The overage during filling accounts for the loss of API while on the shelf or during storage. For example, commercial DEPO-TESTOSTERONE™ is sold as a 200 mg of testosterone cypionate in a vial but includes a 1% overage in the 200 mg vial (i.e., 202 mg of testosterone cypionate) to account for loss of API.

A formulation that is “free of visible crystals,” as used herein, refers to a formulation that has no noticeable crystals upon unenhanced visual inspection.

The term “consisting essentially of,” as used herein means that the composition or formulation referred to necessarily includes the listed ingredients and is open to unlisted ingredients that do not materially affect the basic and novel properties of the composition.

Significant studies have been completed on DEPO-TESTOSTERONE™ to better understand the mechanisms involved in the crystal formation phenomenon in unopened vials. Attempts to nucleate crystal formation using process relevant artificial substrates were unsuccessful. Laboratory findings also suggested that vial defects are not a primary cause of crystal formation. Indirect evidence suggests that undissolved testosterone cypionate particles that are small enough to pass through the sterilizing filter may act as seed crystals. Laboratory studies showed that addition of testosterone cypionate particles result in subsequent crystal growth in the product due to the supersaturation of the solution.

### Example 1: Solubility of Commercially Available DEPO-TESTOSTERONE™ 200 mg/mL

The solubility of the commercially available 200 mg/mL formulation of DEPO-TESTOSTERONE™ (testosterone cypionate) in cottonseed oil was tested and shown to be approximately 186 mg/mL at 20° C.

### Example 2: Solubility of Paternal Formulations of the Invention

A formulation of the invention was made by the following steps. First, 470 mg/mL cottonseed oil, 30% v/v benzyl benzoate, and 9.45 mg/mL benzyl alcohol were added to a tank and mixed to dissolve the benzyl benzoate and benzyl alcohol in the cottonseed oil. Next 200 mg/mL of testosterone cypionate was added to the tank and dissolved. The solution was mixed at 15-35° C., sterilized by filtration using a dry sterile filter assembly fitted with dry sterilizing grade filters into dry sterile receivers. The sterile solution was then aseptically filled into sterile containers and packaged.

The solubility of the formulation of the invention was tested and found to be approximately 233 mg/mL at 20° C. A comparison of the components of the commercial formulation and the formulation of the invention is shown in Table 1.

TABLE 1

| | | | Formulation of the | |
|---|---|---|---|---|
| Ingredients | Commercial Formulation | | Invention | |
| Testosterone Cypionate | 200 | mg | 200 | mg |
| Benzyl benzoate | 0.2 | mL | 0.3 | mL |
| Cottonseed oil | 560.0 | mg | 470.0 | mg |
| Benzyl alcohol | 9.45 | mg | 9.45 | mg |
| Solubility at 20° C. | 186 | mg/mL | 233 | mg/mL |

It was unexpectedly found that improving solubility through increasing the benzyl benzoate content from 20% to 30% (v/v) in the formulation, the potential for crystals to form at low temperatures were eliminated.

In addition, it was unexpectedly found that increasing the level of benzyl benzoate from 20% to 30% (v/v) in the formulation with a reduction in cottonseed oil improves the solubility of the active ingredient in the vehicle, resulting in a product that is not supersaturated at label storage conditions. These changes maintain the 200 mg/mL strength of the formulation while not affecting the drug-in-oil solution characteristics of the formulation.

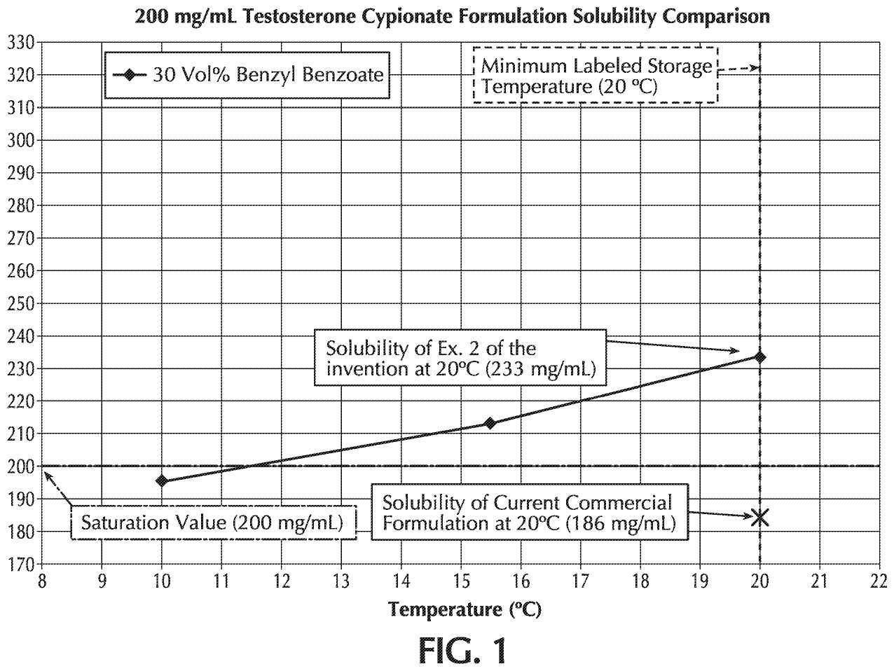

FIG. 1 illustrates the difference in solubility as a function of temperature for the formulation of the present invention and the commercially available formulation. FIG. 1 also shows the saturation value for testosterone cypionate is 200 mg/mL. Any formulation that has a solubility below 200 mg/mL at a given temperature will be supersaturated and therefore be susceptible to crystal formulation. This is the case for the current commercial 200 mg/mL DEPO-TESTOSTERONE™ product at the recommended storage temperature of 20° C., which is 186 mg/mL, as shown in FIG. 1. FIG. 1 shows the comparison of the commercial formulation to the formulation of the invention (i.e., with 30% v/v of benzyl benzoate), which has a solubility above 200 mg/mL at 20° C. (i.e., 233 mg/mL), and therefore is not susceptible to crystal formation at the recommended storage temperature.

### Example 3: Stability of Modified Formulation

Three separate 120 L lots of the formulation of the invention in Example 2 were manufactured. The lots were then filled into 1 mL/2 mL glass vials with the I-Tran stopper and 1 mL/2 mL glass vials with the FM457 stopper and stored at 25° C./60% RH (relative humidity) inverted through 36 months and 40° C./75% RH inverted through 6 months.

All lots met the acceptance criteria through the reported intervals and thus met the shelf-life specifications through 36 months at long-term and 6 months accelerated storage conditions.

1. A pharmaceutical formulation for parental use comprising testosterone cypionate with a concentration of 200 mg/mL, benzyl benzoate with a concentration of 25%-35% v/v, cottonseed oil, and a preservative.

2. The pharmaceutical formulation of claim 1 wherein the benzyl benzoate concentration is 30% v/v, the cottonseed oil has a concentration of 460-480 mg/mL, and the preservative is benzyl alcohol with a concentration of 9 to 10 mg/mL.

3. The pharmaceutical formulation of claim 2 wherein the cottonseed oil has a concentration of 470 mg/mL, and the benzyl alcohol has a concentration of 9.45 mg/mL.

4. The pharmaceutical formulation of claim 3 wherein the formulation has a volume of 1 mL.

5. The formulation of claim 4 consisting essentially of 200 mg of testosterone cypionate, 0.3 mL benzyl benzoate, 470 mg of cottonseed oil, and 9.45 mg benzyl alcohol.

6. The formulation of claim 5, wherein the formulation is free of visible crystals when stored at 20° C.

7. A pharmaceutical product comprising a vial containing a formulation according to claim 6.

8. A kit which comprises the pharmaceutical product according to claim 7 and a package insert that is free of instructions to warm and shake the vial.
